(19) United States
(12) Patent Application Publication
(10) Pub. No.: US 2025/0185996 A1
(43) Pub. Date: Jun. 12, 2025

(54) ADAPTIVE CHANNEL SELECTION FOR DATA COLLECTION DURING ACTIVITY

(71) Applicant: Oura Health Oy, Oulu (FI)

(72) Inventors: Jaakko Tapio Vartiainen, Oulu (FI); Olli Petteri Heikkinen, Oulu (FI)

(21) Appl. No.: 18/954,120

(22) Filed: Nov. 20, 2024

Related U.S. Application Data
(60) Provisional application No. 63/601,484, filed on Nov. 21, 2023.

(51) Int. Cl. A61B 5/00

(52) U.S. Cl. CPC A61B 5/7221; A61B 5/0062; A61B 5/6802

(57) ABSTRACT

Methods, systems, and devices for wearable ring device are described. For example, a system may acquire first physiological data via multiple optical channels of a wearable ring device and may acquire motion data associated with the wearable ring device based on the user performing an activity. The system may input the motion data into a machine learning model to identify one or more optical channels of the multiple optical channels associated with a greatest measurement quality, the machine learning model including a set of measurement quality metrics that are weighted in accordance with historical motion data. In such cases, the set of measurement quality metrics may be weighted based on a correlation between the motion data and at least a subset of the historical motion data. The wearable ring device may collect second physiological data using the one or more identified optical channels based on the user performing the activity.

### CROSS REFERENCE

The present Application for Patent claims the benefit of U.S. Provisional Patent Application No. 63/601,484 by VARTIAINEN et al., entitled “ADAPTIVE CHANNEL SELECTION FOR DATA COLLECTION DURING ACTIVITY,” filed Nov. 21, 2023, assigned to the assignee hereof, and expressly incorporated by reference herein.

### FIELD OF TECHNOLOGY

The following relates to wearable devices and data processing, including adaptive channel selection for data collection during activity.

### BACKGROUND

Some wearable devices may be configured to collect data from users. For example, a wearable device may include one or more sensors that collect physiological data from a user. In some cases, the one or more sensors may be associated with one or more measurement paths, where each of the one or more measurement paths may be associated with a measurement quality and a power consumption.

### BRIEF DESCRIPTION OF THE DRAWINGS

FIG. 1 illustrates an example of a system that supports adaptive channel selection for data collection during activity in accordance with aspects of the present disclosure.

FIG. 2 illustrates an example of a system that supports adaptive channel selection for data collection during activity in accordance with aspects of the present disclosure.

FIG. 3 shows an example of a wearable device that supports adaptive channel selection for data collection during activity in accordance with aspects of the present disclosure.

FIG. 4 shows a block diagram of an apparatus that supports adaptive channel selection for data collection during activity in accordance with aspects of the present disclosure.

FIG. 5 shows a block diagram of a wearable device manager that supports adaptive channel selection for data collection during activity in accordance with aspects of the present disclosure.

FIG. 6 shows a diagram of a system including a device that supports adaptive channel selection for data collection during activity in accordance with aspects of the present disclosure.

FIG. 7 show flowcharts illustrating methods that support adaptive channel selection for data collection during activity in accordance with aspects of the present disclosure.

### DETAILED DESCRIPTION

Wearable devices, such as a wearable ring device, may be used to collect, monitor, and track physiological data associated with a user based on sensor measurements performed by the wearable device. Examples of physiological data may include temperature data, heart rate data, photoplethysmography (PPG) data, blood-oxygen saturation data, and the like. The physiological data collected, monitored, and tracked via the wearable device may be used to gain health insights about the user, such as the user's sleeping patterns, activity patterns, and the like.

Many wearable devices exhibit sensor designs in which the various sensors of the wearable devices are arranged such that multiple measurement paths may exist between one or more sensors of the wearable device. For example, a wearable device designed to be worn on a finger of a user may include a set of sensors (e.g., light-emitting diodes (LEDs) and photodetectors). In this example, the set of sensors may be configured to perform various types of measurements, such as heart rate measurements and blood oxygen saturation measurements. In some cases, the set of sensors may be arranged around the wearable device such that multiple measurement paths, that may be referred to as optical channels or optical paths, may be produced between a pair of sensors to collect physiological data associated with a heart rate of the user and to perform blood oxygen saturation measurements.

However, depending on the arrangement of optical channels (and the positioning of the wearable device relative to the user's skin), some optical channels may facilitate high signal quality for some types of measurements (e.g., heart rate detection), but low signal quality for other types of measurements (e.g., blood oxygen saturation measurements). For example, a single optical channel may utilize pulsating blood vessels to perform high-quality heart rate measurements, but such pulsating blood vessels may be perceived as noise that detrimentally affect blood oxygen saturation measurements. As such, different optical channels may be associated with different measurement qualities. Thus, a system associated with the wearable device may attempt to identify which optical channels result in a greatest measurement quality for a given measurement and select the identified optical channel for data acquisition related to the given measurement.

However, when a user performs an activity, the wearable device may be exposed to external forces that cause the wearable device to move. In such cases, the movement of the wearable device due to the user performing the activity may result in noise that may impact an ability for the system to determine accurate measurement qualities of each optical channel. Thus, the system may be unable to determine which optical channel (e.g., or optical channels) acquire physiological data that is most representative of the actual physiological data (e.g., removes noise due to motion the best). As such, the system may be unable to determine which optical channels to select for acquiring physiological data associated with the user during activity.

Accordingly, the techniques described herein may support adaptive channel selection for data acquisition during activity of a user. In particular, aspects of the present disclosure may support techniques for selecting one or more optical channels of a set of optical channels supported by a wearable device based on motion data associated with the user performing an activity. For example, a wearable device may collect first physiological data associated with a user via a set of optical channels supported by the wearable device, where each optical channel of the set of optical channels is associated with a photodetector and an LED. Additionally, the wearable device (e.g., or another device of a system associated with the wearable device, such as a user device) may collect motion data associated with the wearable device based on the user performing an activity. That is, the wearable device may collect motion data associated with movement (e.g., rotation, sliding, vibration, etc.) of the wearable device due to the user performing the activity.

As such, the wearable device may input the motion data into a machine learning model (e.g., or multiple) to identify one or more optical channels of the set of optical channels to use to collect physiological data associated with the user while the user performs the activity. Specifically, the machine learning model may be built into the wearable device (e.g., and/or the system), where the machine learning model weighs different measurement quality metrics used to calculate a measurement quality of each optical channel based on historical motion data (e.g., based on pre-user testing). That is, pre-user testing may identify that under a first set of motion conditions (e.g., a first subset of the historical motion data), a first subset of the measurement quality metrics may best represent the measurement quality of each optical channel regardless of the motion (e.g., best remove the “noise” from the motion), while under a second set of motion conditions (e.g., a second subset of the historical motion data), a second subset of the measurement quality metrics may best represent the measurement quality of each optical channel regardless of the motion. Thus, during the first set of motion conditions, the machine learning model may weigh the first subset of the measurement quality metrics more heavily, and during the second set of motion conditions, the machine learning model may weigh the second subset of the measurement quality metrics more heavily.

In other words, the system may input the collected motion data (e.g., associated with a set of motion conditions) into the machine learning model and the machine learning model may identify weights associated with the set of measurement quality metrics based on comparing the collected motion data with historical motion data. That is, the machine learning model may correlate, or match, collected motion data to a subset of the historical motion data and may identify the weights associated with the subset of the historical motion data to apply to the set of measurement quality metrics. As such, the machine learning model may calculate a measurement quality (e.g., a cumulative measurement quality metric) for each optical channel of the set of optical channels based on a respective set of measurement quality metrics associated with each optical channel and based on the identified weights. Thus, the machine learning model may identify one or more optical channels of the set of optical channels based on the calculated measurement qualities associated with each optical channel of the set of optical channels. In other words, the machine learning model may identify one or more optical channels of the set of optical channels associated with a greatest measurement quality (e.g., out of the set of optical channels).

As such, the wearable device may acquire second physiological data associated with the user via the one or more identified optical channels while the user performs the activity. Additionally, the system may turn off one or more non-identified optical channels of the set of optical channels based on acquiring the second physiological data via the one or more identified optical channels to reduce power consumption.

In some examples, the machine learning model may identify the weights associated with the set of measurement quality metrics based on identifying the activity being performed by the user. For example, the machine learning model may associate different weights for the set of measurement quality metrics with different activities, such as a first set of weights associated with running, a second set of weights associated with biking, and a third set of weights associated with rowing, among other sets of weights. Additionally, the machine learning model may associate historical motion data with each activity. For example, running may be associated with a first subset of the historical motion data, biking may be associated with a second subset of the historical motion data, and rowing may be associated with a third subset of the historical motion data. As such, the machine learning model may identify an activity that the user is performing based on correlating the collected motion data with a subset of the historical motion data and may identify weights to apply based on the identified activity. Additionally, or alternatively, the wearable device (e.g., or another associated device) may identify the activity that the user is performing based on the motion data and may input an indication of the activity into the machine learning model, such that the machine learning model may identify weights to apply to the set of measurement quality metrics based on the indicated activity.

Additionally, the system may continue to train, or update, the machine learning model based on user-specific historical motion data (e.g., as compared to historical motion data associated with a population of users during pre-user testing). That is, the system may perform “calibration sessions” in which the system collects physiological data associated with the user using all of the optical channels of the set of optical channels while the user performs an activity (e.g., during at least part of the activity). Additionally, the system may identify which optical channels were most representative of the user's physiological data without the effects of noise due to motion. For example, physiological data collected via each optical channel may be associated with a respective frequency and the system may compare the frequencies associated with physiological data collected via each optical channel to a frequency associated with the motion data. Correlation (e.g., alignment) between a frequency of physiological data collected via an optical channel and the frequency associated with the motion data may indicate that the optical channel is representative of the user's physiological data without the effects of noise due to motion.

Additionally, the system may identify which measurement quality metrics would result in selection of said optical channels that are most representative of the user's physiological data. In other words, the system may identify which measurement quality metrics would result in a calculated measurement quality associated with said optical channels being greatest out of the set of optical channels. Thus, the system may modify one or weights associated with the set of measurement quality metrics such that the identified measurement quality metrics are weighted more heavily. Additionally, the system may associate the modified weights with a subset of the historical motion data that corresponds to the activity performed by the user.

Additionally, or alternatively, the system may update the machine learning model based on a comparison of the frequency associated with the motion data to a frequency associated with the second physiological data collected via the identified one or more optical channels. In other words, the system may determine if the second physiological data collected via the one or more optical channels is representative of the user's physiological data without the effects of noise due to motion. The second physiological data may be determined to be representative of the user's physiological data without the effects of noise due to motion based on a difference between the frequency associated with the motion data and the frequency associated with the second physiological data collected via the identified one or more optical channels being less than a threshold. In some cases, the system may input the difference between the frequency associated with the motion data to and the frequency associated with the second physiological data collected via the identified one or more optical channels into the machine learning model, such that the machine learning model may update or modify one or weights associated with the set of measurement quality metrics (e.g., associated with the activity performed by the user) based on the difference.

Aspects of the disclosure are initially described in the context of systems supporting physiological data collection from users via wearable devices. Aspects of the disclosure are further illustrated by and described with reference to apparatus diagrams, system diagrams, and flowcharts that relate to adaptive channel selection for data collection during activity.

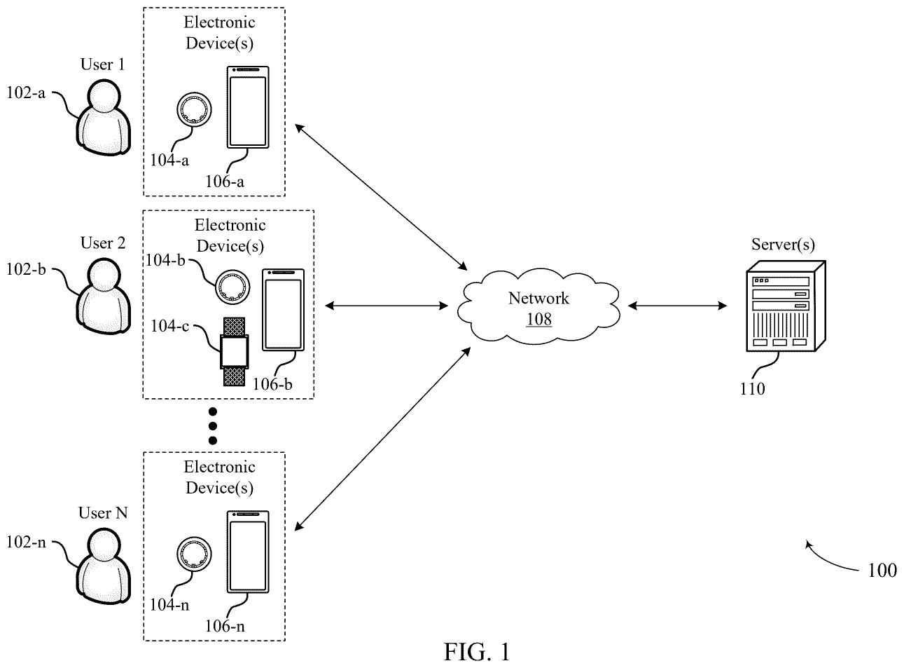

FIG. 1 illustrates an example of a system 100 that supports adaptive channel selection for data collection during activity in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, or blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for adaptive channel selection for data acquisition during activity of a user 102. In particular, aspects of the present disclosure may support techniques for selecting one or more optical channels of a set of optical channels supported by a ring 104 based on motion data associated with the user 102 performing an activity. For example, a ring 104 may collect first physiological data associated with a user 102 via a set of optical channels supported by the ring 104, where each optical channel of the set of optical channels is associated with a photodetector and a LED. Additionally, the ring 104 (e.g., or another device of the system 100 associated with the ring 104, such as a user device 106) may collect motion data associated with the ring 104 based on the user 102 performing an activity. That is, the ring 104 may collect motion data associated with the ring 104 moving or vibrating due to the user 102 performing the activity.

As such, respective devices of the system 100 associated with the ring 104 may input the motion data into a machine learning model (e.g., or multiple) to identify one or more optical channels of the set of optical channels to use to collect physiological data associated with the user 102 while the user 102 performs the activity. Specifically, the machine learning model may be built into the ring 104 (e.g., and/or the system 100), where the machine learning model weights different measurement quality metrics used to calculate a measurement quality associated with each optical channel based on historical motion data. In other words, the system 100 may input the collected motion data into the machine learning model and the machine learning model may identify weights associated with the set of measurement quality metrics based on comparing the collected motion data with historical motion data, where the identified weights are associated with the historical motion data. That is, different subsets of historical motion data may be associated with different weights, thus, the machine learning model may identify a subset of the historical motion data that correlates with the collected motion data and may identify weights associated with the subset of the historical motion data.

As such, the machine learning model may calculate a measurement quality associated with each optical channel of the set of optical channels based on a respective set of measurement quality metrics associated with each optical channel and based on the identified weights. Thus, the machine learning model may identify one or more optical channels of the set of optical channels based on the calculated measurement qualities. In other words, the machine learning model may identify one or more optical channels of the set of optical channels associated with a greatest measurement quality (e.g., out of the set of optical channels). As such, the ring 104 may acquire second physiological data associated with the user 102 via the one or more identified optical channels while the user 102 performs the activity. Additionally, the system 100 may turn off one or more non-identified optical channels of the set of optical channels based on acquiring the second physiological data via the one or more identified optical channels to reduce power consumption.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

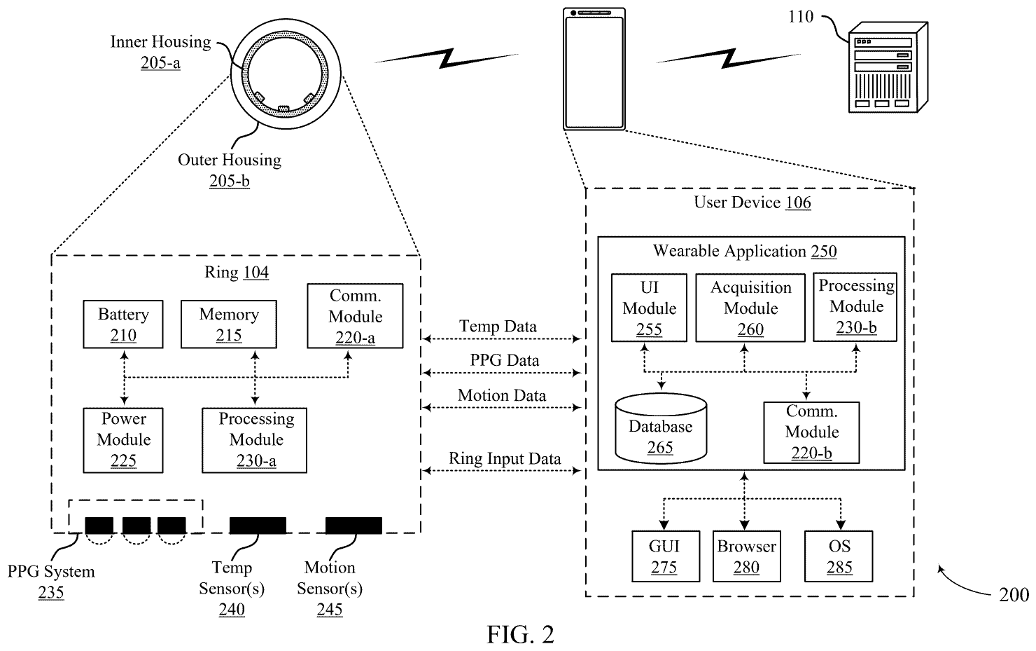

FIG. 2 illustrates an example of a system 200 that supports adaptive channel selection for data collection during activity in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, or software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS) 285, a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, or the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, or timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, or activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques for adaptive channel selection for data acquisition during activity of a user 102. In particular, aspects of the present disclosure may support techniques for selecting one or more optical channels of a set of optical channels supported by a ring 104 based on motion data associated with the user 102 performing an activity. For example, a ring 104 may collect first physiological data associated with a user 102 via a set of optical channels supported by the ring 104. In some cases, the set of optical channels may be associated with a PPG system 235, where each optical channel of the set of optical channels is associated with a photodetector and a LED. Additionally, the ring 104 (e.g., or another device of the system 200 associated with the ring 104, such as a user device 106) may collect motion data associated with the ring 104 via one or more motion sensors 245 based on the user 102 performing an activity. That is, the ring 104 may collect motion data associated with movement of the ring 104 due to the user 102 performing the activity.

As such, respective devices of the system 200 associated with the ring 104 may input the motion data into a machine learning model (e.g., or multiple) to identify one or more optical channels of the set of optical channels to use to collect physiological data associated with the user 102 while the user 102 performs the activity. In some examples, the machine learning model may be built into the ring 104 (e.g., in a processing module 230-a of the ring 104), may be built into the user device 106 associated with the ring 104 (e.g., in a processing module 230-b of the user device 106), or in another device associated with the ring 104 (e.g., connected via the servers 110), or any combination thereof. Specifically, the machine learning model may weight different measurement quality metrics used to calculate a measurement quality associated with each optical channel based on historical motion data. In other words, the system 200 may input the collected motion data into the machine learning model and the machine learning model may identify weights associated with the set of measurement quality metrics based on comparing the collected motion data with historical motion data. That is, different subsets of historical motion data may be associated with different weights, thus, the machine learning model may identify a subset of the historical motion data that correlates with the collected motion data and may identify weights associated with the subset of the historical motion data to apply to the set of measurement quality metrics.

As such, the machine learning model may calculate a measurement quality associated with each optical channel of the set of optical channels based on a respective set of measurement quality metrics associated with each optical channel and based on the identified weights. Thus, the machine learning model may identify one or more optical channels of the set of optical channels based on the calculated measurement qualities. In other words, the machine learning model may identify one or more optical channels of the set of optical channels associated with a greatest measurement quality (e.g., out of the set of optical channels). As such, the ring 104 may acquire second physiological data associated with the user 102 via the one or more identified optical channels while the user 102 performs the activity. Additionally, the system 200 may turn off one or more non-identified optical channels of the set of optical channels based on acquiring the second physiological data via the one or more identified optical channels to reduce power consumption.

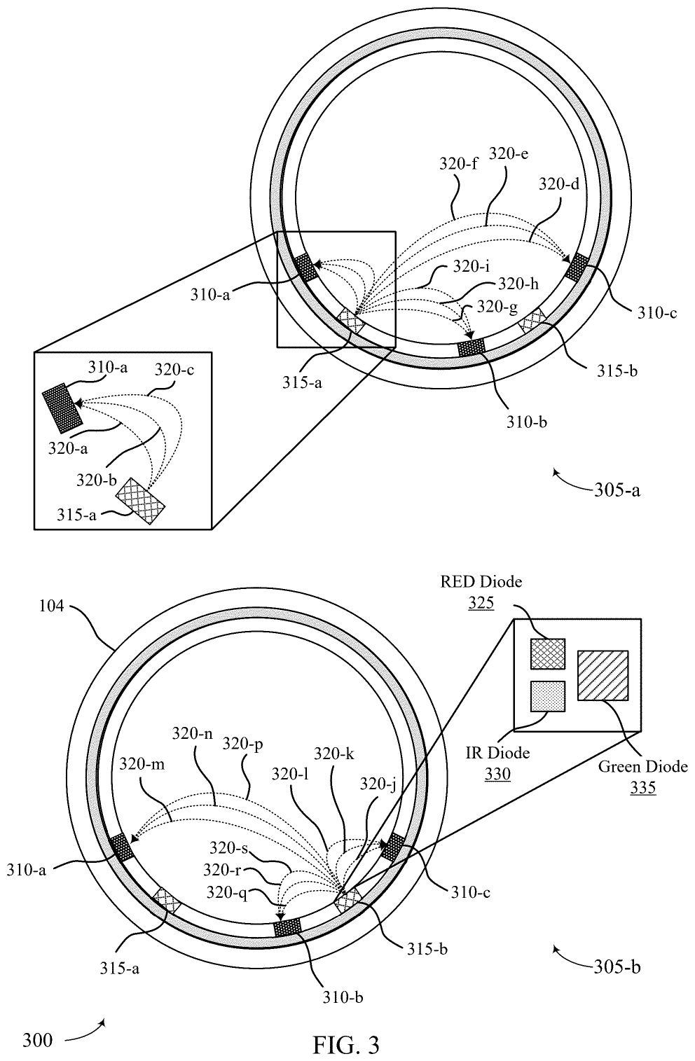

FIG. 3 shows an example of a wearable device 300 that supports adaptive channel selection for data collection during activity in accordance with aspects of the present disclosure. The wearable device 300 may implement, or be implemented by, aspects of the system 100, the system 200, or both.

The wearable device 300 shown in FIG. 3 illustrates an example of a ring 104. The ring 104 may include one or more photodetectors 310, such as a photodetector 310-a (e.g., PD1), a photodetector 310-b (e.g., PD2), and a photodetector 310-c (e.g., PD3), and one or more light-emitting components (e.g., LEDs 315), such as an LED 315-a (e.g., LED1) and an LED 315-b (e.g., LED2), among other electronic components. In some implementations, one or more photodetectors 310, one or more LEDs 315, or both, may be combined as a single component or may be separate components. In some cases, as depicted in cross sectional view 305-a and cross sectional view 305-b, a set of photodetectors 310, a set of LEDs 315, or both, may be located at radial positions within an inner circumference of the ring 104.

In some implementations, some of the sensors (e.g., the photodetector 310-a, the photodetector 310-c, the LEDs 315) of the ring may be positioned on/within the ring 104 symmetrically with respect to an axis of the ring 104, where one or more sensors (e.g., the photodetector 310-b) are positioned asymmetrically with respect to the axis and/or the other sensors. Additionally, each LED 315 may include one or more light-emitting chips or components, such as a red diode (e.g., LED) 325, an IR diode 330, and a green diode 335. Additionally, each diode may be configured to (e.g., be capable of) emitting light within a respective wavelength range. For example, a red diode 325 may emit light within a first wavelength range (e.g., red light), an IR diode 330 may emit light within a second wavelength range (e.g., IR light), and a green diode 335 may emit light within a third wavelength range (e.g., green light). In some cases, the first wavelength range, the second wavelength range, and the third wavelength range may be unique (e.g., different). In this regard, the LEDs 315-a, 315-b may be referred to as “triple-LEDs” that are each configured to emit light in three separate wavelength ranges.

Thus, the ring 104 may support multiple optical channels 320, which may also be referred to as optical paths or measurement paths, of different lengths. That is, an optical channel 320 may be an optical channel 320 between a photodetector 310 and an LED 315 over which light may be transmitted. For example, an optical channel 320-a, an optical channel 320-b, and an optical channel 320-c may exist between the LED 315-a and the photodetector 310-a. That is, the optical channel 320-a may be associated with emission of light (e.g., within the third wavelength range) from a green diode 335 on the LED 315-a, the optical channel 320-b may be associated with emission of light (e.g., within the first wavelength range) from a red diode 325 on the LED 315-a, and the optical channel 320-c may be associated with emission of light (e.g., within the second wavelength range) from an IR diode 330 on the LED 315-a.

Similarly, an optical channel 320-d, an optical channel 320-e, and an optical channel 320-f may exist between the LED 315-a and the 310-c, where the optical channel 320-d is associated with the green diode 335 on the LED 315-a, the optical channel 320-e is associated with the red diode 325 on the LED 315-a, and the optical channel 320-f is associated with the IR diode 330 on the LED 315-a. Additionally, an optical channel 320-g, an optical channel 320-h, and an optical channel 320-i may exist between the LED 315-a and the 310-b, where the optical channel 320-g is associated with the green diode 335 on the LED 315-a, the optical channel 320-h is associated with the red diode 325 on the LED 315-a, and the optical channel 320-i is associated with the IR diode 330 on the LED 315-a.

Similarly, an optical channel 320-j, an optical channel 320-k, and an optical channel 320-l may exist between the LED 315-b and the 310-c, where the optical channel 320-j is associated with a green diode 335 on the LED 315-b, the optical channel 320-k is associated with a red diode 325 on the LED 315-b, and the optical channel 320-l is associated with an IR diode 330 on the LED 315-b. Additionally, the optical channel 320-m, an optical channel 320-n, and an optical channel 320-p may exist between the LED 315-b and the 310-a, where the optical channel 320-m is associated with the green diode 335 on the LED 315-b, the optical channel 320-n is associated with the red diode 325 on the LED 315-b, and the optical channel 320-p is associated with the IR diode 330 on the LED 315-b. Additionally, the optical channel 320-q, an optical channel 320-r, and an optical channel 320-s may exist between the LED 315-b and the 310-b, where the optical channel 320-q is associated with the green diode 335 on the LED 315-b, the optical channel 320-r is associated with the red diode 325 on the LED 315-b, and the optical channel 320-s is associated with the IR diode 330 on the LED 315-b

In some cases, a system associated with the ring 104 may collect physiological data associated with the user 102 based on light received by the photodetectors 310 from the LEDs 315 along the optical channels 320. For example, a controller communicatively coupled to (e.g., connected to) one or more of the LEDs 315, or one or more of the photodetectors 310, or any combination thereof, may collect physiological data associated with the user 102 based on light received by a photodetector 310 and light emitted from one or more LEDs 315 (e.g., along one or more optical channels 320).

In some cases, the controller may selectively activate one or more of the LEDs 315 (e.g., diodes, such as a red diode 325, an IR diode 330, or a green diode 335, within the LEDs 315) or photodetectors 310 based on a respective metrics associated with each optical channel 320 (e.g., associated with light received by one or more of the photodetectors 310). That is, the system may determine respective measurement quality metrics associated with each optical channel 320 used to collect physiological data from the user 102 (e.g., from the 18 optical channels supported by the ring 104) and may select one or more optical channels 320 from the set of optical channels 320 to use to collect additional physiological data based on a comparison of the respective measurement quality metrics associated with each optical channel 320.

However, when a user 102 performs an activity, the ring 104 may be exposed to external forces that cause the ring 104 to move (e.g., rotate, vibrate, slide along a finger of the user 102, bounce, etc.). In such cases, the movement of the ring 104 may result in noise that may impact an ability for the system to determine accurate measurement qualities of each optical channel. In other words, the noise may impact one or more measurement quality metrics associated with each optical channel 320 and, in some cases, may impact the one or more measurement quality metrics differently for each optical channel 320 (e.g., depending on the movement caused). As such, the system may be unable to determine which optical channel 320 (e.g., or optical channels 320) is associated with a greatest measurement quality due to the impacts of the noise (e.g., due to the user 102 performing an activity that results in movement of the ring 104). In other words, the system may be unable to determine which optical channel 320 (e.g., or optical channels 320) acquires physiological data that is most representative of the actual physiological data (e.g., removes noise due to motion the best, is impacted by the noise the least). Thus, the system may be unable to determine which optical channel 320 (e.g., or optical channels 320) to select for acquiring physiological data associated with the user 102 during activity.

Accordingly, the system associated with the ring 104 may support adaptive optical channel selection for data acquisition during activity of a user 102. In particular, aspects of the present disclosure may support techniques for selecting one or more optical channels 320 of the multiple optical channels 320 supported by a ring 104 based on motion data associated with the user 102 performing an activity. For example, the ring 104 may collect first physiological data associated with the user 102 via the multiple optical channels 320 supported by the ring 104 (e.g., or a subset of the multiple optical channels 320). Additionally, the ring 104 (e.g., or another device of a system associated with the ring 104, such as a user device 106) may collect motion data associated with the ring 104 based on the user 102 performing an activity. That is, the wearable device may collect motion data associated with movement (e.g., rotation, sliding, vibration, etc.) of the ring 104 due to the user 102 performing the activity.

As such, respective devices of the system associated with the ring 104 may input the motion data into a machine learning model (e.g., or multiple) to identify one or more optical channels 320 of the multiple optical channels 320 to use to collect physiological data associated with the user 102 while the user 102 performs the activity. In some examples, the machine learning model may be built into (e.g., supported by) the ring 104, may be built into the user device 106 associated with the ring 104, or may be built into another device associated with the ring 104 (e.g., connected via servers 110), or any combination thereof. Additionally, or alternatively, the system may input the collected motion data into the machine learning model based on the collected motion data satisfying a threshold.

Specifically, the machine learning model may weight different measurement quality metrics associated with the multiple optical channels 320 based on the collected motion data relative to historical motion data (e.g., based on pre-user testing). In other words, the machine learning model may calculate a measurement quality associated with each optical channel 320 of the multiple optical channels 320 based on multiple measurement quality metrics. However, as described previously, different measurement quality metrics may be affected by motion of the ring 104 in different ways depending on motion of the ring 104, such that some measurement quality metrics are highly impacted by noise from motion of the ring 104 while other measurement quality metrics are minorly impacted by the noise from motion of the ring 104. Further, the impact of the noise on the measurement quality metrics may differ between different movement of the ring 104 (e.g., differ between the type or magnitude of the noise itself). As such, under certain motion conditions, different measurement quality metrics may be considered more heavily due to the said measurement quality metrics being impacted less by the noise. Thus, the machine learning model may weight each measurement quality metric differently based on the motion of the ring 104 (e.g., based on the motion data). In other words, the machine learning model may correlate, or associated, different motion conditions (e.g., different motion data) with different weightings of the measurement quality metrics, where the different motion conditions are based on historical motion data.

For example, pre-user testing may identify that under a first set of motion conditions (e.g., a first subset of the historical motion data), a first subset of the measurement quality metrics may best represent the measurement quality of each optical channel 320 regardless of the motion (e.g., best remove the “noise” from the motion), while under a second set of motion conditions (e.g., a second subset of the historical motion data), a second subset of the measurement quality metrics may best represent the measurement quality of each optical channel 320 regardless of the motion. Thus, during the first set of motion conditions, the machine learning model may weigh the first subset of the measurement quality metrics more heavily, and during the second set of motion conditions, the machine learning model may weigh the second subset of the measurement quality metrics more heavily.

In other words, the system may input the collected motion data (e.g., associated with a set of motion conditions) into the machine learning model and the machine learning model may compare the collected motion data with historical motion data. Further, the system may correlate, or match, the collected motion data to a subset of the historical motion data (e.g., within a threshold tolerance) and may identify weights associated with the subset of the historical motion data to be applied to the measurement quality metrics based on the collected motion data matching the subset of the historical motion data.

As such, the machine learning model may calculate a measurement quality (e.g., overall measurement quality) of each optical channel 320 of the multiple optical channels 320 based on a respective set of measurement quality metrics collected via each optical channel 320 and based on the identified weights (e.g., associated with the subset of the historical motion data). Thus, the machine learning model may identify one or more optical channels 320 of the multiple optical channels 320 based on the calculated measurement qualities (e.g., overall measurement qualities output by the machine learning model) associated with each optical channel 320 of the multiple optical channels 320. In other words, the machine learning model may identify one or more optical channels 320 of the multiple optical channels 320 associated with a greatest measurement quality (e.g., out of the set of optical channels).

As such, the ring 104 may acquire second physiological data associated with the user 102 via the one or more identified optical channels 320 while the user 102 performs the activity. Additionally, the system may turn off one or more non-identified optical channels 320 of the multiple optical channels 320 based on acquiring the second physiological data via the one or more identified optical channels 320 to reduce power consumption.

In some examples, the measurement quality metrics used to calculate the measurement quality of an optical channel 320 may be based on any combination of ambient light (e.g., inward vs outward pointing photodetectors 310), PPG direct current (DC) levels, spectrogram-based metrics, high frequency noise (e.g., heart rate is in the band 30-220 BPM and anything above that may be high frequency noise), gyroscope-based metrics, a quantity of harmonics, motion-based heart rate (e.g., as compared to optical-based heart rate), differences between an ACM or gyroscope spectrogram and a PPG spectrogram, temperature (e.g., high finger temperature indicates good PPG quality), and contact pressure (e.g., relative to optimal contact pressure).

In some examples, the machine learning model may identify the weights associated with the measurement quality metrics based on identifying the activity being performed by the user 102. For example, the machine learning model may associate different weights for the set of measurement quality metrics with different activities, such as a first set of weights associated with running, a second set of weights associated with biking, and a third set of weights associated with rowing, among other sets of weights associated with other activities. Additionally, the machine learning model may associate historical motion data with each activity. For example, running may be associated with a first subset of the historical motion data, biking may be associated with a second subset of the historical motion data, and rowing may be associated with a third subset of the historical motion data. As such, the machine learning model may identify an activity that the user is performing based on correlating the collected motion data with a subset of the historical motion data and may identify weights to apply to the measurement quality metrics based on the identified activity. For example, the machine learning model may identify that the collected motion data correlates to the first subset of historical motion data and, thus, the user is running. As such, the machine learning model may apply the first set of weights to the measurement quality metrics based on the user 102 running.

Additionally, or alternatively, the ring 104 (e.g., or another associated device) may identify the activity that the user 102 is performing based on the collected motion data and may input an indication of the activity into the machine learning model, such that the machine learning model may identify weights for the set of measurement quality metrics based on the indicated activity. For example, the ring 104 may identify that the user 102 is biking based on the collected motion data and may input an indication of biking into the machine learning model. As such, the machine learning model may select the second set of weights associated with biking for application to the measurement quality metrics. In some examples, the ring 104 may identify the activity based on correlation between the collected motion data and historical motion data associated with the activity. Additionally, or alternatively, the system may identify the activity based on a user input (e.g., via the user device 106), or tag, of the activity.

Additionally, the system may continue to train, or update, the machine learning model based on user-specific historical motion data (e.g., as compared to historical motion data associated with a population of users 102 during pre-user testing). That is, the system may perform “calibration sessions” in which the system collects physiological data associated with the user 102 while the user performs an activity (e.g., during at least part of the activity) using all of the optical channel 320 of the multiple optical channels 320. Additionally, the system may identify which optical channels 320 are most representative of the user's physiological data without the effects of noise due to motion. For example, physiological data collected via each optical channel 320 may be associated with a respective frequency and the system may compare the frequencies associated with physiological data collected via each optical channel 320 to a frequency associated with the motion data. Correlation (e.g., alignment) between a frequency of physiological data collected via an optical channel 320 and the frequency associated with the motion data may indicate that the optical channel 320 is representative of the user's physiological data without the effects of noise due to motion.

Additionally, the system may identify which measurement quality metrics would result in selection of said optical channels 320 that are most representative of the user's physiological data. In other words, the system may identify which measurement quality metrics would result in a calculated measurement quality associated with said optical channels 320 being greatest out of the multiple optical channels 320. Thus, the system may modify one or weights associated with the set of measurement quality metrics such that the identified measurement quality metrics are weighted more heavily. Additionally, the system may associate the modified weights with a subset of the historical motion data that corresponds to the activity performed by the user 102.

Additionally, or alternatively, the system may update the machine learning model based on a comparison of the frequency associated with the motion data to a frequency associated with the second physiological data collected via the identified one or more optical channels 320. In other words, the system may determine if the second physiological data collected via the one or more optical channels 320 is representative of the user's physiological data without the effects of noise due to motion. The second physiological data may be determined to be representative of the user's physiological data without the effects of noise due to motion based on a difference between the frequency associated with the motion data and a frequency associated with the second physiological data collected via the identified one or more optical channels 320 being less than a threshold. In some cases, the system may input the difference between the frequency associated with the motion data to and the frequency associated with the second physiological data collected via the identified one or more optical channels 320 into the machine learning model, such that the machine learning model may update or modify one or weights associated with the measurement quality metrics (e.g., associated with the activity performed by the user 102) based on the difference.

Though described in the context of the ring 104 including the photodetector 310-a, the photodetector 310-b, and the photodetector 310-c, as well as the LED 315-a and the LED 315-b, this is merely an exemplary depiction as is not to be regarded as a limitation of the present disclosure. In this regard, the ring 104 may support any quantity of optical channels 320 (e.g., optical paths) generated by any quantity of photodetectors 310 and LEDs 315. Further, the photodetectors 310 and the LEDs 315 are not limited to the positions depicted in FIG. 3.

Additionally, though described in the context of red diodes 325, IR diodes 330, and green diodes 335, it is understood that diodes on an LED 315 may be associated with any color of light within a spectrum. That is, a diode may be configured to emit light within a wavelength range not limited to the first wavelength range, the second wavelength range, or the third wavelength range. For example, as described previously herein, light-emitting components of the present disclosure (e.g., LEDs 315) may include additional diodes configured to emit light in any wavelength range of color, such as yellow light, blue light, etc.

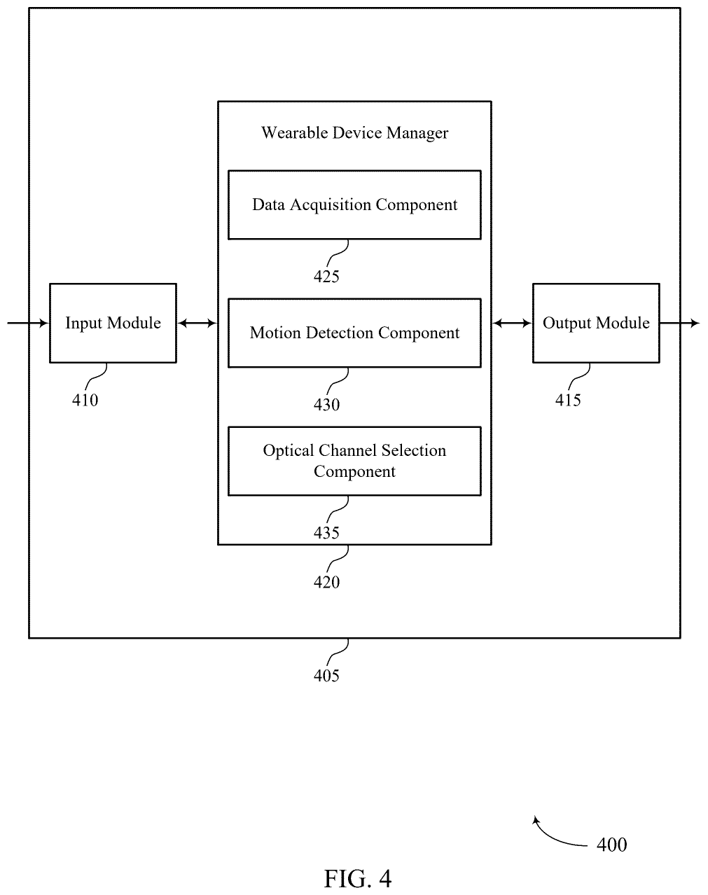

FIG. 4 shows a block diagram 400 of a device 405 that supports adaptive channel selection for data collection during activity in accordance with aspects of the present disclosure. The device 405 may include an input module 410, an output module 415, and a wearable device manager 420. The device 405, or one or more components of the device 405 (e.g., the input module 410, the output module 415, and the wearable device manager 420), may include at least one processor, which may be coupled with at least one memory, to support the described techniques. Each of these components may be in communication with one another (e.g., via one or more buses).

For example, the wearable device manager 420 may include a data acquisition component 425, a motion detection component 430, or an optical channel selection component 435, or any combination thereof. In some examples, the wearable device manager 420, or various components thereof, may be configured to perform various operations (e.g., receiving, monitoring, transmitting) using or otherwise in cooperation with the input module 410, the output module 415, or both. For example, the wearable device manager 420 may receive information from the input module 410, send information to the output module 415, or be integrated in combination with the input module 410, the output module 415, or both to receive information, transmit information, or perform various other operations as described herein.

The data acquisition component 425 may be configured as or otherwise support a means for acquiring first physiological data from a user via a plurality of optical channels of a wearable device, wherein each optical channel comprises a light-emitting component and a photodetector. The motion detection component 430 may be configured as or otherwise support a means for acquiring motion data associated with the wearable device based at least in part on the user performing an activity. The optical channel selection component 435 may be configured as or otherwise support a means for inputting the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data. The data acquisition component 425 may be configured as or otherwise support a means for acquiring second physiological data using the one or more identified optical channels based at least in part on the user performing the activity.

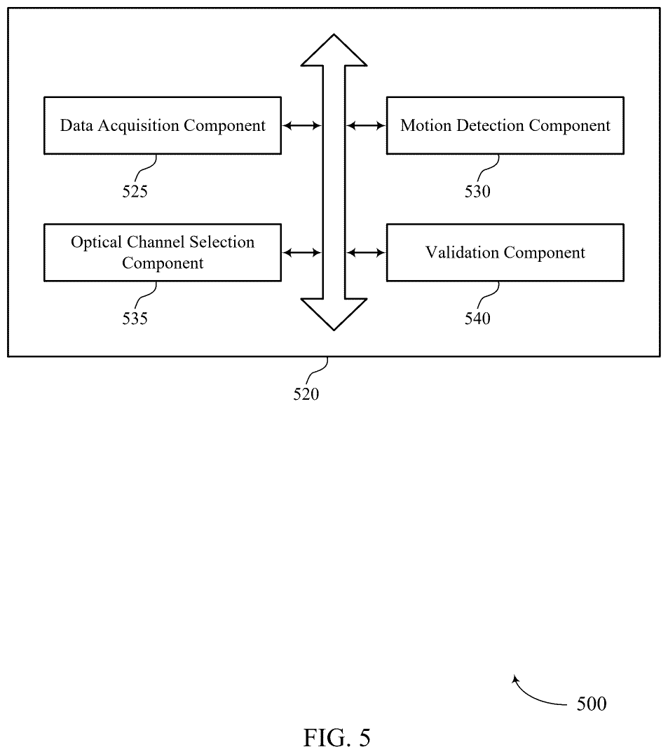

FIG. 5 shows a block diagram 500 of a wearable device manager 520 that supports adaptive channel selection for data collection during activity in accordance with aspects of the present disclosure. The wearable device manager 520 may be an example of aspects of a wearable device manager or a wearable device manager 420, or both, as described herein. The wearable device manager 520, or various components thereof, may be an example of means for performing various aspects of adaptive channel selection for data collection during activity as described herein. For example, the wearable device manager 520 may include a data acquisition component 525, a motion detection component 530, an optical channel selection component 535, or a validation component 540, or any combination thereof. Each of these components, or components of subcomponents thereof (e.g., one or more processors, one or more memories), may communicate, directly or indirectly, with one another (e.g., via one or more buses).

The data acquisition component 525 may be configured as or otherwise support a means for acquiring first physiological data from a user via a plurality of optical channels of a wearable device, wherein each optical channel comprises a light-emitting component and a photodetector. The motion detection component 530 may be configured as or otherwise support a means for acquiring motion data associated with the wearable device based at least in part on the user performing an activity. The optical channel selection component 535 may be configured as or otherwise support a means for inputting the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data. In some examples, the data acquisition component 525 may be configured as or otherwise support a means for acquiring second physiological data using the one or more identified optical channels based at least in part on the user performing the activity.

In some examples, the data acquisition component 525 may be configured as or otherwise support a means for turning off one or more non-identified optical channels of the plurality of optical channels based at least in part on acquiring the second physiological data using the one or more identified optical channels.

In some examples, the motion detection component 530 may be configured as or otherwise support a means for acquiring additional motion data associated with the wearable device. In some examples, the motion detection component 530 may be configured as or otherwise support a means for identifying the user is no longer performing the activity based at least in part on the additional motion data. In some examples, the data acquisition component 525 may be configured as or otherwise support a means for acquiring third physiological data from the user via the plurality of optical channels of the wearable device based at least in part on the user no longer performing the activity.

In some examples, the motion detection component 530 may be configured as or otherwise support a means for acquiring second motion data associated with the wearable device based at least in part on the user performing a second activity. In some examples, the data acquisition component 525 may be configured as or otherwise support a means for acquiring third physiological data using each of the plurality of optical channels during the second activity. In some examples, the optical channel selection component 535 may be configured as or otherwise support a means for updating the one or more machine learning models based at least in part on a respective quality of the third physiological data associated with each of the plurality of optical channels and based at least in part on the second motion data.

In some examples, the historical motion data is associated with a population of users, where the population of users is associated with one or more same characteristics associated with the user.

In some examples, the validation component 540 may be configured as or otherwise support a means for comparing a first frequency associated with the motion data to a second frequency associated with the second physiological data. In some examples, the optical channel selection component 535 may be configured as or otherwise support a means for updating the one or more machine learning models based at least in part on a difference between the first frequency and the second frequency satisfying a threshold.

In some examples, the validation component 540 may be configured as or otherwise support a means for comparing a first frequency associated with the motion data to a second frequency associated with the first physiological data, wherein inputting the motion data into the one or more machine learning models is based at least in part on a difference between the first frequency and the second frequency satisfying a threshold.

In some examples, inputting the motion data into the one or more machine learning models is based at least in part on the motion data satisfying a threshold, the user inputting a tag associated with the activity, or both.

In some examples, the set of measurement quality metrics are associated with one or more of an ACM frequency, a gyroscopic frequency, a PPG frequency, a PPG DC level, an ambient light level, a frequency of noise, an accelerometer-based heart rate, a contact pressure, and a temperature.

In some examples, the set of measurement quality metrics are weighted based at least in part on a type of the activity being performed.

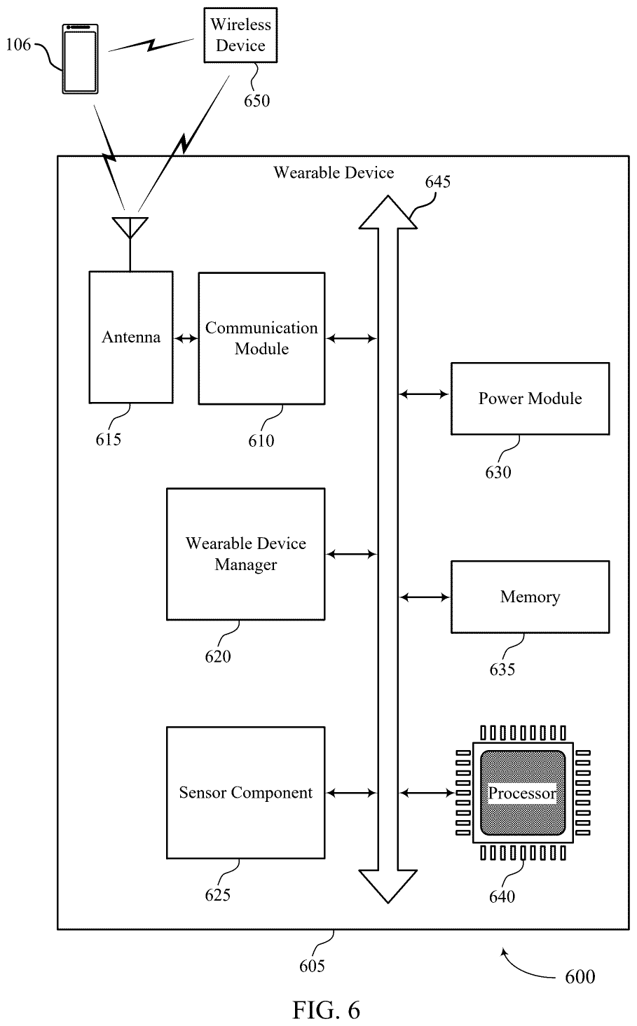

FIG. 6 shows a diagram of a system 600 including a device 605 that supports adaptive channel selection for data collection during activity in accordance with aspects of the present disclosure. The device 605 may be an example of or include the components of a device 405 as described herein. The device 605 may include an example of a wearable device 104, as described previously herein. The device 605 may include components for bi-directional communications including components for transmitting and receiving communications with a user device 106 and a server 110, such as a wearable device manager 620, a communication module 610, an antenna 615, a sensor component 625, a power module 630, at least one memory 635, at least one processor 640, and a wireless device 650. These components may be in electronic communication or otherwise coupled (e.g., operatively, communicatively, functionally, electronically, electrically) via one or more buses (e.g., a bus 645).

For example, the wearable device manager 620 may be configured as or otherwise support a means for acquiring first physiological data from a user via a plurality of optical channels of a wearable device, wherein each optical channel comprises a light-emitting component and a photodetector. The wearable device manager 620 may be configured as or otherwise support a means for acquiring motion data associated with the wearable device based at least in part on the user performing an activity. The wearable device manager 620 may be configured as or otherwise support a means for inputting the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data. The wearable device manager 620 may be configured as or otherwise support a means for acquiring second physiological data using the one or more identified optical channels based at least in part on the user performing the activity.

By including or configuring the wearable device manager 620 in accordance with examples as described herein, the device 605 may support techniques for adaptive channel selection during user activity which may result in reduced power consumption and increased measurement quality (e.g., more accurate measurements).

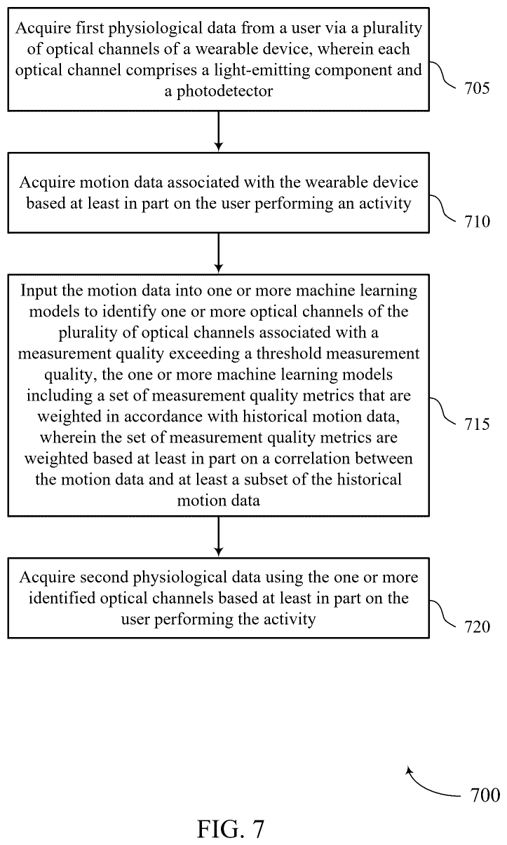

FIG. 7 shows a flowchart illustrating a method 700 that supports adaptive channel selection for data collection during activity in accordance with aspects of the present disclosure. The operations of the method 700 may be implemented by a wearable device or its components as described herein. For example, the operations of the method 700 may be performed by a wearable device as described with reference to FIGS. 1 through 6. In some examples, a wearable device may execute a set of instructions to control the functional elements of the wearable device to perform the described functions. Additionally, or alternatively, the wearable device may perform aspects of the described functions using special-purpose hardware.

At 705, the method may include acquiring first physiological data from a user via a plurality of optical channels of a wearable device, wherein each optical channel comprises a light-emitting component and a photodetector. The operations of block 705 may be performed in accordance with examples as disclosed herein. In some examples, aspects of the operations of 705 may be performed by a data acquisition component 525 as described with reference to FIG. 5.

At 710, the method may include acquiring motion data associated with the wearable device based at least in part on the user performing an activity. The operations of block 710 may be performed in accordance with examples as disclosed herein. In some examples, aspects of the operations of 710 may be performed by a motion detection component 530 as described with reference to FIG. 5.

At 715, the method may include inputting the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data. The operations of block 715 may be performed in accordance with examples as disclosed herein. In some examples, aspects of the operations of 715 may be performed by an optical channel selection component 535 as described with reference to FIG. 5.

At 720, the method may include acquiring second physiological data using the one or more identified optical channels based at least in part on the user performing the activity. The operations of block 720 may be performed in accordance with examples as disclosed herein. In some examples, aspects of the operations of 720 may be performed by a data acquisition component 525 as described with reference to FIG. 5.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include acquiring first physiological data from a user via a plurality of optical channels of a wearable device, wherein each optical channel comprises a light-emitting component and a photodetector, acquiring motion data associated with the wearable device based at least in part on the user performing an activity, inputting the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data, and acquiring second physiological data using the one or more identified optical channels based at least in part on the user performing the activity.

An apparatus is described. The apparatus may include one or more memories storing processor executable code, and one or more processors coupled with the one or more memories. The one or more processors may individually or collectively operable to execute the code to cause the apparatus to acquire first physiological data from a user via a plurality of optical channels of a wearable device, wherein each optical channel comprises a light-emitting component and a photodetector, acquire motion data associated with the wearable device based at least in part on the user performing an activity, input the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data, and acquire second physiological data using the one or more identified optical channels based at least in part on the user performing the activity.

Another apparatus is described. The apparatus may include means for acquiring first physiological data from a user via a plurality of optical channels of a wearable device, wherein each optical channel comprises a light-emitting component and a photodetector, means for acquiring motion data associated with the wearable device based at least in part on the user performing an activity, means for inputting the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data, and means for acquiring second physiological data using the one or more identified optical channels based at least in part on the user performing the activity.

A non-transitory computer-readable medium storing code is described. The code may include instructions executable by a processor to acquire first physiological data from a user via a plurality of optical channels of a wearable device, wherein each optical channel comprises a light-emitting component and a photodetector, acquire motion data associated with the wearable device based at least in part on the user performing an activity, input the motion data into one or more machine learning models to identify one or more optical channels of the plurality of optical channels associated with a greatest measurement quality, the one or more machine learning models including a set of measurement quality metrics that are weighted in accordance with historical motion data, wherein the set of measurement quality metrics are weighted based at least in part on a correlation between the motion data and at least a subset of the historical motion data, and acquire second physiological data using the one or more identified optical channels based at least in part on the user performing the activity.

Some examples of the method, apparatus, and non-transitory computer-readable medium described herein may further include operations, features, means, or instructions for turning off one or more non-identified optical channels of the plurality of optical channels based at least in part on acquiring the second physiological data using the one or more identified optical channels.

Some examples of the method, apparatus, and non-transitory computer-readable medium described herein may further include operations, features, means, or instructions for acquiring additional motion data associated with the wearable device, identifying the user may be no longer performing the activity based at least in part on the additional motion data, and acquiring third physiological data from the user via the plurality of optical channels of the wearable device based at least in part on the user no longer performing the activity.

Some examples of the method, apparatus, and non-transitory computer-readable medium described herein may further include operations, features, means, or instructions for acquiring second motion data associated with the wearable device based at least in part on the user performing a second activity, acquiring third physiological data using each of the plurality of optical channels during the second activity, and updating the one or more machine learning models based at least in part on a respective quality of the third physiological data associated with each of the plurality of optical channels and based at least in part on the second motion data.

In some examples of the method, apparatus, and non-transitory computer-readable medium described herein, the historical motion data may be associated with a population of users, where the population of users may be associated with one or more same characteristics associated with the user.

Some examples of the method, apparatus, and non-transitory computer-readable medium described herein may further include operations, features, means, or instructions for comparing a first frequency associated with the motion data to a second frequency associated with the second physiological data and updating the one or more machine learning models based at least in part on a difference between the first frequency and the second frequency satisfying a threshold.

Some examples of the method, apparatus, and non-transitory computer-readable medium described herein may further include operations, features, means, or instructions for comparing a first frequency associated with the motion data to a second frequency associated with the first physiological data, wherein inputting the motion data into the one or more machine learning models may be based at least in part on a difference between the first frequency and the second frequency satisfying a threshold.

Some examples of the method, apparatus, and non-transitory computer-readable medium described herein may further include operations, features, means, or instructions for inputting the motion data into the one or more machine learning models may be based at least in part on the motion data satisfying a threshold, the user inputting a tag associated with the activity, or both.

In some examples of the method, apparatus, and non-transitory computer-readable medium described herein, the set of measurement quality metrics may be associated with one or more of an ACM frequency, a gyroscopic frequency, a PPG frequency, a PPG DC level, an ambient light level, a frequency of noise, an accelerometer-based heart rate, a contact pressure, and a temperature.

In some examples of the method, apparatus, and non-transitory computer-readable medium described herein, the set of measurement quality metrics may be weighted based at least in part on a type of the activity being performed.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, or optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, or discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, or firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method, comprising:
acquiring first physiological data from a user via a plurality of optical channels of a wearable device, wherein each optical channel comprises a light-emitting component and a photodetector, and wherein each optical channel of the plurality of optical channels is associated with a respective set of measurement quality metrics based at least in part on the first physiological data;
acquiring motion data associated with the wearable device based at least in part on the user performing an activity;
identifying, via one or more machine learning models, a set of weights based at least in part on a correlation between the motion data and first historical motion data from a plurality of historical motion data;
identifying, via the one or more machine learning models, one or more optical channels of the plurality of optical channels associated with a greatest measurement quality based at least in part on the set of weights and based at least in part on the respective set of measurement quality metrics associated with each optical channel of the plurality of optical channels; and
acquiring second physiological data using the one or more optical channels of the plurality of optical channels associated with the greatest measurement quality based at least in part on the user performing the activity.

2. The method of claim 1, further comprising:
turning off a respective light-emitting component, a respective photodetector, or both, associated with one or more second optical channels of the plurality of optical channels other than the one or more optical channels associated with the greatest measurement quality based at least in part on acquiring the second physiological data using the one or more optical channels associated with the greatest measurement quality.

3. The method of claim 1, further comprising:
acquiring additional motion data associated with the wearable device;
identifying the user is no longer performing the activity based at least in part on the additional motion data; and
acquiring third physiological data from the user via the plurality of optical channels of the wearable device based at least in part on the user no longer performing the activity.

4. The method of claim 1, further comprising:
acquiring second motion data associated with the wearable device based at least in part on the user performing a second activity;
acquiring third physiological data using each optical channel of the plurality of optical channels during the second activity; and
updating the one or more machine learning models based at least in part on a respective measurement quality associated with each optical channel of the plurality of optical channels and based at least in part on the second motion data.

5. The method of claim 1, wherein the plurality of historical motion data is associated with a population of users, where the population of users is associated with one or more same characteristics associated with the user.

6. The method of claim 1, further comprising:
comparing a first frequency associated with the motion data to a second frequency associated with the second physiological data; and
updating the one or more machine learning models based at least in part on a difference between the first frequency and the second frequency satisfying a threshold.

7. The method of claim 1, further comprising:
comparing a first frequency associated with the motion data to a second frequency associated with the first physiological data, wherein inputting the motion data into the one or more machine learning models is based at least in part on a difference between the first frequency and the second frequency satisfying a threshold.

8. The method of claim 1, wherein inputting the motion data into the one or more machine learning models is based at least in part on the motion data satisfying a threshold, the user inputting a tag associated with the activity, or both.

9. The method of claim 1, wherein each respective set of measurement quality metrics is associated with one or more of an analysis calibration model (ACM) frequency, a gyroscopic frequency, a photoplethysmogram (PPG) frequency, a PPG direct current (DC) level, an ambient light level, a frequency of noise, an accelerometer-based heart rate, a contact pressure, and a temperature.

10. The method of claim 1, wherein the set of weights are based at least in part on a type of the activity being performed.

11. The method of claim 1, further comprising:
generating, using the one or more machine learning models, a cumulative measurement quality metric associated with each optical channel of the plurality of optical channels based at least in part on application of the set of weights to the respective set of measurement quality metrics associated with each optical channel of the plurality of optical channels, wherein the greatest measurement quality is associated with a greatest cumulative measurement quality metric.

12. A system, comprising:
a wearable device comprising one or more sensors configured to acquire physiological data from a user; and
one or more processors communicatively coupled with the wearable device, wherein the one or more processors are configured to:
acquire, via the wearable device, first physiological data from the user via a plurality of optical channels, wherein each optical channel comprises a light-emitting component and a photodetector, and wherein each optical channel of the plurality of optical channels is associated with a respective set of measurement quality metrics based at least in part on the first physiological data;
acquire, via the wearable device, motion data associated with the wearable device based at least in part on the user performing an activity;
identify, via one or more machine learning models, a set of weights based at least in part on a correlation between the motion data and first historical motion data from a plurality of historical motion data;
identify, via the one or more machine learning models, one or more optical channels of the plurality of optical channels associated with a greatest measurement quality based at least in part on the set of weights and based at least in part on the respective set of measurement quality metrics associated with each optical channel of the plurality of optical channels; and
acquire, via the wearable device, second physiological data using the one or more optical channels of the plurality of optical channels associated with the greatest measurement quality based at least in part on the user performing the activity.

13. The system of claim 12, wherein the one or more processors are individually or collectively further operable to cause the system to:
turn off a respective light-emitting component, a respective photodetector, or both, associated with one or more second optical channels of the plurality of optical channels other than the one or more optical channels associated with the greatest measurement quality based at least in part on acquiring the second physiological data using the one or more optical channels associated with the greatest measurement quality.

14. The system of claim 12, wherein the one or more processors are individually or collectively further operable to cause the system to:
acquire additional motion data associated with the wearable device;
identify the user is no longer performing the activity based at least in part on the additional motion data; and
acquire third physiological data from the user via the plurality of optical channels of the wearable device based at least in part on the user no longer performing the activity.

15. The system of claim 12, wherein the one or more processors are individually or collectively further operable to cause the system to:
acquire second motion data associated with the wearable device based at least in part on the user performing a second activity;
acquire third physiological data using each optical channel of the plurality of optical channels during the second activity; and
update the one or more machine learning models based at least in part on a respective measurement quality associated with each optical channel of the plurality of optical channels and based at least in part on the second motion data.

16. The system of claim 12, wherein the plurality of historical motion data is associated with a population of users, where the population of users is associated with one or more same characteristics associated with the user.

17. The system of claim 12, wherein the one or more processors are individually or collectively further operable to cause the system to:
compare a first frequency associated with the motion data to a second frequency associated with the second physiological data; and
update the one or more machine learning models based at least in part on a difference between the first frequency and the second frequency satisfying a threshold.

18. The system of claim 12, wherein the one or more processors are individually or collectively further operable to cause the system to:
compare a first frequency associated with the motion data to a second frequency associated with the first physiological data, wherein inputting the motion data into the one or more machine learning models is based at least in part on a difference between the first frequency and the second frequency satisfying a threshold.

19. The system of claim 12, wherein inputting the motion data into the one or more machine learning models is based at least in part on the motion data satisfying a threshold, the user inputting a tag associated with the activity, or both.

20. The system of claim 12, wherein the one or more processors are individually or collectively further operable to cause the system to:
generate, using the one or more machine learning models, a cumulative measurement quality metric associated with each optical channel of the plurality of optical channels based at least in part on application of the set of weights to the respective set of measurement quality metrics associated with each optical channel of the plurality of optical channels, wherein the greatest measurement quality is associated with a greatest cumulative measurement quality metric.
